(19) United States
(12) Patent Application Publication
(10) Pub. No.: US 2011/0158958 A1
(43) Pub. Date: Jun. 30, 2011

(54) Methods for treating ophthalmic disorders, diseases and injuries

(76) Inventor: George L. Sing, New York, NY (US)

(21) Appl. No.: 12/928,234

(22) Filed: Dec. 7, 2010

Related U.S. Application Data
(60) Provisional application No. 61/283,706, filed on Dec. 7, 2009.

(51) Int. Cl. A61K 35/50; A61K 38/19; A61P 27/02

(52) U.S. Cl. 424/93.7; 424/583; 514/20.8

(57) ABSTRACT

The invention is directed to methods for treating ophthalmic disorders, diseases and injuries. In particular, the invention is directed to treating disorders, diseases and injuries of the cornea and ocular surface. Such methods utilize novel compositions including, but not limited to, trophic factor secreting extraembryonic cells (herein referred to as TSE cells), including, but not limited to, Amnion-derived Multipotent Progenitor cells (herein referred to as AMP cells) and conditioned media derived therefrom (herein referred to as Amnion-derived Cellular Cytokine Solution or ACCS), and Physiologic Cytokine Solution (herein referred to as PCS), each alone or in combination with each other and/or other agents.

### CROSS-REFERENCE TO RELATED APPLICATIONS

This application claims priority under 35 USC §119(e) of U.S. Provisional Application No. 61/283,706, filed Dec. 7, 2009, the entirety of which is incorporated herein by reference.

### FIELD OF THE INVENTION

The field of the invention is directed to methods for treating ophthalmic disorders, diseases and injuries. In particular, the field of the invention is directed to treating disorders, diseases and injuries of the cornea and ocular surface. The field of the invention is also directed to treating retinal disorders including injuries, defects and diseases of the retina. Such methods utilize novel compositions including, but not limited to, trophic factor secreting extraembryonic cells (herein referred to as TSE cells), including, but not limited to, Amnion-derived Multipotent Progenitor cells (herein referred to as AMP cells) and conditioned media derived therefrom (herein referred to as Amnion-derived Cellular Cytokine Solution or ACCS), and Physiologic Cytokine Solution (herein referred to as PCS), each alone or in combination with each other and/or other agents.

### DESCRIPTION OF RELATED ART

Ahmed, S., et al, (Stem Cells, 2007, January 25 e-publication) studied the differentiation of human embryonic stem cells into corneal epithelial like cells by in vitro replication of the corneal epithelial stem cell niche and reported that culturing human embryonic stem cells on collagen IV using medium conditioned by limbal fibroblasts results in the loss of pluripotency and differentiation into epithelial like cells.

Tejwani, S., et al. (Cornea, 2007, 26(1):21-6) performed a retrospective review of case records of patients who had undergone amniotic membrane transplantation (AMT) for chemical and thermal injuries to the ocular surface and determined that AMT helps ocular surface reconstruction, promotes rapid epithelial healing and partially restores limbal stem cell function.

Uchida, S., et al., (Neuroscience Letters, 2003, 341:1-4) reported that factors secreted by human amniotic epithelial cells promote the survival of rat retinal ganglion cells.

Chacko, D. M., et al, (Biochem Biophy Res Commun, 2000, 268(3):842-6) studied the survival and differentiation of cultured retinal progenitors transplanted in the subretinal space of the rat and concluded that the cultured retinal progenitors can be a viable reagent for therapeutic transplantation.

Otani, A., et al, (J Clin Invest, 2004, 114(6):765-7) studied the rescue of retinal degeneration by intravitreally injected adult bone marrow-derived lineage-negative hematopoietic stem cells and reported that whenever a fraction of adult bone marrow-derived stem cells (lineage-negative hematopoietic stem cells) containing endothelial precursors stabilized and rescued retinal blood vessels that would have ordinarily completely degenerated, a dramatic neurotrophic rescue was also observed.

Smith, L. E. (J Clin Invest, 2004, 114(6):755-7) studied bone marrow-derived stem cells' ability to preserve cone vision in retinitis pigmentosa and found that such cells prevent cone loss.

### BACKGROUND OF THE INVENTION

There are several major ophthalmological disorders, diseases and injuries that affect the cornea, lens and retina. Serious corneal disorders, diseases and injuries include corneal ulcers, corneal wounds (i.e. thermal, chemical, physical, surgical), keratitis (inflammation of the cornea), and dry eye syndrome (Sjogren's syndrome). Serious lens disorders include cataracts and refractive errors. The most serious disorders and diseases of the retina include macular holes, retinal degeneration, diabetic retinopathy, retinal ischemia, Retinitis Pigmentosa, Usher syndrome, Stargardt disease, retinal detachment, choroideremia, and retinoschisis.

The cornea is the transparent front part of the eye that covers the iris, pupil, and anterior chamber. The human cornea has five layers. From the anterior to posterior the five layers of the human cornea are the 1) corneal epithelium, a thin layer of stratified squamous epithelium which are fast-growing and easily-regenerated cells that are kept moist with tears. The corneal epithelium is continuous with the conjunctival epithelium which is composed of about 6 layers of cells which are shed constantly and are regenerated by cell division in the basal layer. 2) Bowman's layer which is a tough layer of condensed collagen fibers that protects the corneal stroma, which consists of similar irregularly arranged collagen fibers; 3) The corneal stroma which is a thick, transparent middle layer, consisting of regularly arranged collagen fibers along with sparsely distributed interconnected keratocytes, which are the cells for general repair and maintenance; 4) Descemet's membrane which is a thin acellular layer that serves as the modified basement membrane of the corneal endothelium, from which the cells are derived; and 5) the corneal endothelium which is a simple squamous or low cuboidal monolayer of mitochondria-rich cells responsible for regulating fluid and solute transport between the aqueous and corneal stromal compartments.

The lens is a transparent, biconvex structure in the eye that, along with the cornea, helps to refract light to be focused on the retina. The lens has three main parts: the lens capsule, the lens epithelium, and the lens fibers. The lens capsule forms the outermost layer of the lens and the lens fibers form the bulk of the interior of the lens. The cells of the lens epithelium, located between the lens capsule and the outermost layer of lens fibers, are found only on the anterior side of the lens.

The retina is a very thin layer of light-sensitive neural tissue lining at the inner posterior surface of the eyeball. It is composed of six classes of neurons and one type of glial cell that are interconnected in a highly organized structure. The rod and cone photoreceptor cells reside in the outer nuclear layer; the horizontal, bipolar, and amacrine interneurons plus the Muller glial cells reside in the inner nuclear layer; and the ganglion and displaced amacrine cells reside in the ganglion cell layer. The major function of the retina is to convert light signals detected by photoreceptor cells into electrical impulses, which are then transmitted to the brain via the optic nerve which is derived from the projecting axons of the ganglion cells. Any loss and/or damage of the various retinal cell types will result in disruption of the normal transmission of nerve impulses and lead to impaired vision.

Many ophthalmic disorders, diseases and injuries are treated with surgery. In some instances, treatment has focused on gene therapy to correct inheritable disorders such as those found in Retinitis Pigmentosa. Other areas of treatment and current research are directed towards evaluating the role of growth factors and/or cytokines in preventing or protecting against retinal cell death or generating new retinal cells to replace lost ones. Similar studies with growth factors and/or cytokines aim to protect and/or regenerate limbal stem cells to treat corneal injuries such as corneal wounds. Still other treatment approaches use various inhibitors of neovascularization (i.e. VEGF inhibitors) to prevent or reduce the amount of new blood vessel growth in the eye and associated vascular leak and hemorrhage such as that seen in diabetic retinopathy and age-related macular degeneration. Other treatment options for corneal disorders/diseases/injuries include antibiotics, antifungals or antivirals if infection is present; mitomycin C; topical steroids to treat inflammation; bandage contact lens; fibrin glue; tarsorraphy (partial suturing of the eyelids); autologous serum; Gunderson flap; and corneal transplant.

A promising new area of research is directed to evaluating the potential of stem cells to replace damaged or lost retinal cells or corneal epithelial cells, including limbal stem cells (see, for example, Chacko, D. M., et al, (Biochem Biophy Res Commun 2000, 268(3):842-6); Otani, A., et al, (J Clin Invest 2004 114(6):765-7); Smith, L. E. (J Clin Invest 2004 114(6):755-7; Ahmed, S., et al, (Stem Cells, 2007, January 25 e-publication). Also being studied is retinal transplantation (see Ng, T. F., et al, Chem Immunol Allergy, 2007, 92:300-16).

To date, no treatment option exists that is able to completely ameliorate corneal epithelial or retinal damage, provide neuroprotection to retinal cells, or induce the growth and development of new corneal epithelial cells or limbal stem cells or retinal cells to replace damaged or dead cells, any or all of which could help return the patient to normal or near normal visual function. Therefore, it is an object of the instant invention to provide such treatment options for patients suffering from ophthalmic disorders, diseases and injuries, in particular, corneal disorders, diseases and injuries.

### BRIEF SUMMARY OF THE INVENTION

It is an object of the instant invention to provide novel methods for treating ophthalmic disorders, diseases and injuries including treating corneal disorders/diseases/injuries such as keratitis (inflammation of the cornea), corneal burns, dry eye syndrome, etc. It is also an object of the instant invention to provide novel methods for treating ophthalmic disorders including retinal disorders, diseases and injuries. Such methods for treating ophthalmic disorders/diseases/injuries utilize novel compositions including trophic factor secreting extraembryonic cells (herein referred to as TSE cells), including Amnion-derived Multipotent Progenitor (AMP) cells, conditioned media and/or cell products derived therefrom (herein referred to as Amnion-derived Cellular Cytokine Solution or ACCS), and Physiologic Cytokine Solution (herein referred to as PCS), each alone and/or in combination with each other and/or with other agents including active and/or inactive agents.

Accordingly, a first aspect of the invention is a method for treating an ophthalmic disorder, disease or injury in a patient in need thereof comprising administering to the patient a therapeutically effective amount of one or more compositions comprising TSE cells, conditioned media derived therefrom, cell lysate derived therefrom, cell products derived therefrom, or PCS. In specific embodiments the ophthalmic disorder, disease or injury is a corneal disorder or a lens disorder.

A second aspect of the invention is a method for stimulating proliferation or regeneration of corneal epithelial cells in a patient in need thereof comprising administering to the patient a therapeutically effective amount of one or more compositions comprising TSE cells, conditioned media derived therefrom, cell lysate derived therefrom, cell products derived therefrom, or PCS.

A third aspect of the invention is a method for stimulating proliferation and/or activation of corneal stromal fibroblasts in a patient in need thereof comprising administering to the patient a therapeutically effective amount of one or more compositions comprising TSE cells, conditioned media derived therefrom, cell lysate derived therefrom, cell products derived therefrom, or PCS.

A fourth aspect of the invention is a method for treating an ocular surface disorder, disease or injury in a patient in need thereof comprising administering to the patient a therapeutically effective amount of one or more compositions comprising TSE cells, conditioned media derived therefrom, cell lysate derived therefrom, cell products derived therefrom, or PCS. A particular embodiment of aspect four is one wherein the ocular surface disorder, disease or injury is keratitis, corneal ulcers or corneal wounds. In specific embodiments the corneal wounds are chemical wounds, thermal wounds, surgical wounds, or mechanical wounds. Another specific embodiment is one wherein the keratitis is caused by amoebic, bacterial, fungal or viral infection, photokeratitis, exposure (eyelid dysfunction), chemical injury, trauma, surgery (laser-assisted in situ keratomileusis (LASIK), photorefractive keratectomy (PRK), cataract, corneal transplant, pterygium surgery), keratoconus, Fuchs' dystrophy, or keratoconjunctivitis sicca.

A fifth aspect of the invention is a method for reducing the risk for corneal transplant rejection in a patient in need thereof comprising administering to the patient a therapeutically effective amount of one or more compositions comprising TSE cells, conditioned media derived therefrom, cell lysate derived therefrom, cell products derived therefrom, or PCS.

In particular embodiments of aspects one through five the TSE cells are AMP cells. In a more specific embodiment the AMP cells. In other more specific embodiments of aspects one through five the conditioned medium is Amnion-derived Cellular Cytokine Solution (herein referred to as ACCS), including pooled ACCS. In still another specific embodiment the ACCS or pooled ACCS is formulated for sustained-release.

In additional particular embodiments of aspects one through five the TSE cells, including AMP cells, the conditioned media derived therefrom, cell lysate derived therefrom or cell products derived therefrom are administered in combination with other agents or treatment modalities. In more specific embodiments the other agents are active agents. And in the most specific embodiments the active agents are growth factors, cytokines, inhibitors, immunosuppressive agents, steroids, chemokines, antibodies, antibiotics, antifungals, antivirals, mitomycin C, or other cell types. Another specific embodiment in one in which the other treatment modalities are bandage contact lens, fibrin glue, tarsorraphy (partial suturing of the eyelids), autologous serum, Gunderson flap or corneal transplant.

A sixth aspect of the invention is a method of treating retinal disorders, diseases and injuries. In a specific embodiment the retinal disorders, diseases and injuries include macular holes, retinal detachment, retinal degeneration or diabetic retinopathy. In another particular embodiment the retinal degeneration is macular degeneration, Retinitis Pigmentosa, choroideremia or retinoschisis. In yet another particular embodiment the macular degeneration is dry age-related macular degeneration, wet age-related macular degeneration or juvenile-onset macular degeneration.

A seventh aspect of the invention is a method for stimulating regeneration of retinal cells in a patient in need thereof comprising administering to the patient a therapeutically effective amount of one or more compositions comprising TSE cells, including AMP cells, conditioned media derived therefrom, cell lysate derived therefrom or cell products derived therefrom, or PCS. In one embodiment the retinal cells are photoreceptor cells, including rod cells and cones cells.

An eighth aspect of the invention is a method for preventing or ameliorating retinal cell degeneration in a patient in need thereof comprising administering to the patient a therapeutically effective amount of one or more compositions comprising TSE cells, including AMP cells, conditioned media derived therefrom, cell lysate derived therefrom or cell products derived therefrom, or PCS. In one embodiment the retinal cells are photoreceptor cells, including rod cells and cones cells.

A ninth aspect of the invention is a method for preventing retinal transplant rejection in a patient in need thereof comprising administering to the patient a therapeutically effective amount of one or more compositions comprising TSE cells, including AMP cells, conditioned media derived therefrom, cell lysate derived therefrom or cell products derived therefrom, or PCS.

In specific embodiments of aspects six through nine, the TSE cells are AMP cells. In still another specific embodiment the conditioned media is ACCS. And in yet another specific embodiment the ACCS is pooled ACCS. In other specific embodiments the AMP cells and ACCS are co-administered, either simultaneously (AMP cells and ACCS) or consecutively (i.e. AMP cells then ACCS or ACCS then AMP cells). In still other specific embodiments the AMP cells and/or ACCS is administered or co-administered one or more times In another specific embodiment of aspects six through nine of the invention, the TSE cells, including AMP cells, conditioned media derived therefrom, cell lysate derived therefrom or cell products derived therefrom, or PCS, are administered in combination with other agents. In certain embodiments the other agents are active agents. In particular embodiments the active agents are neuroprotective agents, growth factors, cytokines, inhibitors, chemokines, antibodies, antibiotics, antifungals, antivirals, immunosuppressive agents or other cell types. In a particular embodiment the inhibitor is a VEGF inhibitor. Examples of VEGF inhibitors include VEGF Trap® (in development by Regeneron Pharmaceuticals, Inc.), Macugen® (EyeTech Pharmaceuticals), Avastin® (Genentech) and Lucentis® (Genentech). In another particular embodiment the immunosuppressive agents are cyclosporine, methotrexate, FK-506 and corticosteroids. In another particular embodiment the other cell types are retinal progenitor cells (see, for example, Coles, B. L., et al., PNAS USA 2004, 101(44):15772-7.). In another particular embodiment, the TSE cells, including AMP cells, conditioned media derived therefrom, cell lysate derived therefrom or cell products derived therefrom, or PCS, are administered in combination with gene therapy treatment.

Other features and advantages of the invention will be apparent from the accompanying description, examples and the claims. The contents of all references, pending patent applications and issued patents, cited throughout this application are hereby expressly incorporated by reference. In case of conflict, the present specification, including definitions, will control.

### DEFINITIONS

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cells” or “EE cells” means a population of cells derived from the extraembryonic tissue.

As used herein, the term “trophic factor secreting extraembryonic cells” or “TSE cells” means a population of cells derived from the extraembryonic tissue which have the characteristic of secreting VEGF, Angiogenin, PDGF and TGFfβ2 and the MMP inhibitors TIMP-1 and/or TIMP-2 at physiologically relevant levels in a physiologically relevant temporal manner into the extracellular space or into the surrounding culture media. TSE cells have not been cultured in the presence of any non-human animal materials, making them and cell products derived from them suitable for human clinical use as they are not xeno-contaminated. TSE cells may be selected from populations of cells and compositions described in this application and in US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179, the contents of which are incorporated herein by reference in their entirety.

As used herein, the term “Amnion-derived Multipotent Progenitor cell” or “AMP cell” means a specific population of cells that are epithelial cells derived from the amnion. AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 μg/mL for PDGF, ˜2.5-2.7 ng/mL for TGFβ2, ˜0.68 μmL for TIMP-1 and ˜1.04 μg/mL for TIMP-2. The AMP cells may optionally express Thymosin β4. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion-derived cells, from which AMP cells are isolated, will not react with antibodies to the stem/progenitor cell markers c-kit (CD 117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of amnion epithelial cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of amnion epithelial cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the term “differentiation” means the process by which cells become progressively more specialized.

As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein.

As used herein, the term “Amnion-derived Cellular Cytokine Solution” or “ACCS” means conditioned medium that has been derived from AMP cells that have been cultured in basal media supplemented with human serum albumin. ACCS has previously been referred to as “amnion-derived cellular cytokine suspension”.

The term “physiological level” as used herein means the level that a substance in a living system is found and that is relevant to the proper functioning of a biochemical and/or biological process.

As used herein, the term “Physiologic Cytokine Solution” or “PCS” composition means a composition which is not cell-derived and which has physiologic concentrations of one or more factors selected from VEGF, Angiogenin, PDGF and TGFβ2 and at least one MMP inhibitor. Examples of suitable MMP inhibitors include but are not limited to TIMP-1 and TIMP-2. Details on PCS can be found in U.S. Publication No. US-2009-0054339-A1, the contents of which is incorporated herein by reference.

As used herein, the term “pooled” means a plurality of compositions that have been combined to create a new composition having more constant or consistent characteristics as compared to the non-pooled compositions.

The term “therapeutically effective amount” means that amount of a therapeutic agent necessary to achieve a desired physiological effect (i.e. promote corneal healing).

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases. In some instances, it may be desirable to lyse the cells and retain the cellular membrane portion and discard the remaining portion of the lysed cells.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells, including a cell suspension or cells incorporated into a matrix or tissue, that are either in an undifferentiated, partially differentiated, or fully differentiated form into a human or other animal.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “neurodegeneration” means the progressive loss of neurons in the central nervous system. This includes but is not limited to immediate loss of neurons due to injury or disease followed by subsequent loss of connecting or adjacent neurons. One example of neurodegeneration is retinal degeneration, in which the cells of the retina (i.e. photoreceptors known as rods and cones) are progressively lost.

As used herein, the term “neuroprotection” means the arrest and/or reverse progression of neurodegeneration following a central nervous system injury or as a result of disease.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The term “ophthalmically acceptable” with respect to a formulation, composition or ingredient as used herein means having no persistent effect that is substantially detrimental to the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984,“Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Therapeutic Uses

Disorders/Diseases/Injuries of the Cornea

Keratitis refers to inflammation of the cornea. Causes include but are not limited to amoebic, bacterial, fungal or viral infection, photokeratitis, exposure (eyelid dysfunction), chemical injury, trauma, surgery (LASIK, PRK, cataract, corneal transplant, pterygium surgery), or congenital causes such as keratoconus, Fuchs' dystrophy, or keratoconjunctivitis sicca. The compositions and methods of the present invention may be effective in treating corneal inflammation.

Corneal ulcers form when the surface of the cornea is damaged or compromised in some way. The ulcers may be sterile or infected and determines the course of treatment. Bacterially infected ulcers tend to be extremely painful and are typically associated with a break in the epithelium, the outermost layer of the cornea. Certain types of bacteria, such as Pseudomonas, are extremely aggressive and can cause severe damage and even blindness within 24-48 hours if left untreated. Sterile ulcers cause little if any pain. They are often found near the peripheral edge of the cornea and are not necessarily accompanied by a break in the epithelial layer of the cornea. There are many causes of corneal ulcers. Contact lens wearers are at an increased risk of corneal ulcers if they are not diligent in the cleaning, handling, and disinfection of their lenses and cases. Bacterially infected ulcers are also associated with diseases that compromise the corneal surface, creating a window of opportunity for organisms to infect the cornea. Patients with severely dry eyes, who have difficulty blinking, or are unable to care for themselves, are also at risk. Other causes of ulcers include herpes simplex viral infections, inflammatory diseases, corneal abrasions or injuries, and other systemic diseases. The compositions and methods of the present invention may be effective in treating corneal ulcers.

Corneal wounds are injuries to the ocular surface and can be thermal (i.e. burn), chemical (i.e. acid), physical (i.e. abrasions) or surgical wounds (i.e. corneal transplant) or a combination thereof. The compositions and methods of the present invention may be effective in treating corneal wounds.

Dry eye syndrome is one of the most common problems treated by eye physicians. It is usually caused by a problem with the quality of the tear film that lubricates the eyes. Tears are comprised of three layers. The mucus layer coats the cornea, forming a foundation so the tear film can adhere to the eye, the middle aqueous layer provides moisture and supplies oxygen and other important nutrients to the cornea, and the outer lipid layer is an oily film that seals the tear film on the eye and helps to prevent evaporation. Tears are formed by several glands around the eye. The water layer is produced in the lacriminal gland located under the upper eyelid and several smaller glands in the lids make the oil and mucus layers. With each blink, the eyelids spread the tears over the eye. Excess tears flow into two tiny drainage ducts in the corner of the eye by the nose. These ducts lead to tiny canals that connect to the nasal passage. Dry eye syndrome has many causes. One of the most common reasons for dryness is the normal aging process. Many other factors, such as hot, dry or windy climates, high altitudes, air-conditioning and cigarette smoke also cause dry eyes. Many people also find their eyes become irritated when reading or working on a computer. Contact lens wearers may also suffer from dryness because the contacts absorb the tear film, causing proteins to form on the surface of the lens. Certain medications, thyroid conditions, vitamin A deficiency, menopause and diseases such as Parkinson's and Sjogren's can also cause dryness. The compositions and methods of the present invention may be effective in treating dry eye syndrome.

Corneal transplantation. The compositions and methods of the invention may be useful in preventing corneal transplant rejection. U.S. Provisional Application Nos. 60/880,745 and 60/902,440 (incorporated herein by reference) describe in detail the immunosuppressive properties of the compositions of the invention. As stated above, it has been discovered that extraembryonic cells (EE cells) including but not limited to extraembryonic HLA-G positive cells (EHP cells), TSE cells and AMP cells, and/or cell lysates and/or conditioned media derived therefrom,alone or in combination with each other and/or other suitable active agents, are useful agents capable of treating HVG, GVHD, as well as many other immune and/or inflammatory diseases and disorders. The cells express HLA-G, do not express MHC Class II antigens, are telomerase negative, do not form teratomas, are not immortal, secrete cellular modulatory factors, and are readily available in great numbers.

Retinal Disorders

Macular holes (also called macular cysts, retinal holes, retinal tears, and retinal perforations) may occur for a variety of reasons, but are usually a result of traction from the vitreous gel on the macula. Since the macula is responsible for central vision, this problem causes severe and often complete loss of central vision. It is possible for anyone to develop a macular hole, but they are most common among women about 60-70 years of age. Macular holes are typically treated with a surgical technique called transpars plana vitrectomy, which removes the vitreous and replaces it with an air/gas bubble to hold the retina in place while the hole is repaired. Eventually, the body replaces the air/gas bubble with natural fluids. Unfortunately, the surgery itself may permanently damage central vision. Current methods for treating macular holes improve vision in only 40% of eyes. The compositions and methods of the present invention may be effective in treating macular holes.

Retinal detachment occurs when the retina's sensory and pigment layers separate. Because it can cause devastating damage to the vision if left untreated, retinal detachment is considered an ocular emergency that requires immediate medical attention and surgery. There are three types of retinal detachments. The most common type occurs when there is a break in the sensory layer of the retina, and fluid seeps underneath, causing the layers of the retina to separate. The second most common type occurs when strands of vitreous or scar tissue create traction on the retina, pulling it loose. Patients with diabetes are more likely to experience this type. The third type happens when fluid collects underneath the layers of the retina, causing it to separate from the back wall of the eye. This type usually occurs in conjunction with another disease affecting the eye that causes swelling or bleeding. The compositions and methods of the present invention may be effective in treating retinal detachment.

Retinal degeneration occurs when the photoreceptor cells (rods and cones) are progressively lost due to disease or injury. There are many types of retinal degeneration including Age-Related Macular Degeneration (AMD), which can be either the more common “dry” form or the less common, but more serious, “wet” form. Stargardt disease is an inherited juvenile macular degeneration disorder. Dry AMD cannot be cured, but patients with the condition should continue to remain under an ophthalmologist's care to monitor the affected eye. Also, if the other eye is healthy, screening still should continue, to stay on the lookout for problems. Wet AMD may be successfully treated with laser surgery. However, successful treatment may not mean restoring normal vision, but rather, preventing vision loss from worsening. One drawback of laser surgery is that it may damage some of the neighboring retinal tissue. There are several surgical procedures that may be used depending on the size and type of the abnormal blood vessels. One surgical procedure, called laser photocoagulation, destroys leaking blood vessels that have grown under the macula and halts the damage. A newer laser procedure called photodynamic therapy uses a different laser to treat abnormal blood vessels and a medication injected into the patient's arm. This medication travels through the bloodstream and attaches itself to the abnormal blood vessels, so when the laser light is shown in the eye, the blood vessels alone are destroyed. Both of these procedures must be done before the abnormal blood vessels leak and cause irreversible damage to the retina. Also, because more blood vessels could grow later on, patients who get this treatment need to continue to have follow-up evaluations. In addition to surgery, several new drugs are on the market or in development to treat macular degeneration. These include VEGF inhibitors and other types of molecules. The compositions and methods of the present invention may be effective in treating retinal degeneration.

Retinitis Pigmentosa refers to a group of inherited retinal degeneration disorders. The most common feature of all forms of Retinitis Pigmentosa is the gradual degeneration of the rods and cones. Most forms of RP first cause the degeneration of rod cells. These forms of Retinitis Pigmentosa, sometimes called rod-cone dystrophy, usually begin with night blindness. Patients with Retinitis Pigmentosa cannot adjust well to dark and dimly lit environments. As the disease progresses and more rod cells degenerate, patients lose their peripheral vision. Patients with Retinitis Pigmentosa often experience a ring of vision loss in their mid-periphery with small islands of vision in their very far periphery. Others report the sensation of tunnel vision, as though they see the world through a straw. Many patients with Retinitis Pigmentosa retain a small degree of central vision throughout their life. Usher syndrome is a type of Retinitis Pigmentosa that is also associated with hearing loss. Unfortunately, no clinically significant treatment currently exists for Retinitis Pigmentosa, although much research in the field of gene therapy in underway. The compositions and methods of the present invention may be effective in treating Retinitis Pigmentosa.

Light-induced retinal degeneration includes, but is not limited to, medical-light induced retinal degeneration. Some RP patients are more sensitive to light damage than others (see Paskowitz, D. M., et al., (Br J Ophthalmol 2006;90:1060-1066). Protecting such patients by treatment with the compositions of the invention prior to medical invention utilizing potentially damaging light is contemplated by the methods of the invention.

Choroideremia is a rare inherited disorder that causes progressive loss of vision due to degeneration of the choroid and retina. Formerly called tapetochoroidal dystrophy, choroideremia occurs almost exclusively in males. In childhood, night blindness is the most common first symptom. As the disease progresses, there is loss of peripheral vision or “tunnel vision”, and later a loss of central vision. Progression of the disease continues throughout the individual's life, although both the rate and the degree of visual loss can vary, even within the same family. Vision loss due to choroideremia is caused by degeneration of several layers of cells that are essential to sight. These layers, which line the inside of the back of the eye, are called the choroids, the retinal pigment epithelium and the photoreceptors. The retinal pigment epithelium and the choroid initially deteriorate to cause choroideremia. Eventually, the photoreceptors break down as well. The compositions and methods of the present invention may be effective in treating choroideremia.

Retinoschisis is a rare eye disorder characterized by the abnormal splitting of the retina's sensory layers, resulting in loss of visual function. It is estimated that retinoschisis affects one in 5,000 to 25,000 individuals, primarily young males. Treatment is often aimed at restricting any worsening of the separation so that it does not encroach on the macula. Retinoschisis causes acuity loss in the center of the visual field through the formation of tiny cysts in the retina. The cysts are usually only detectable by a trained clinician. Vision cannot be improved by corrective lenses, as the nerve tissue itself is damaged by these cysts. The compositions and methods of the present invention may be effective in treating retinoschisis.

Diabetic retinopathy occurs as a complication of diabetes. Types of diabetic retinopathy include background diabetic retinopathy, pre-proliferative diabetic retinopathy, clinically significant macular edema and proliferative diabetic retinopathy. Diabetic retinopathy is characterized by vitreous or retinal hemorrhage, retinal microaneurysm, retinal neovascularization and macular edema. During the first three stages of diabetic retinopathy, no treatment is needed, unless macular edema is present. To prevent progression of diabetic retinopathy, diabetics should control their levels of blood sugar, blood pressure, and blood cholesterol. Proliferative retinopathy is treated with laser surgery called scatter laser treatment. Scatter laser treatment helps to shrink the abnormal blood vessels. Because a high number of laser burns are necessary, two or more sessions usually are required to complete treatment. Scatter laser treatment works better before the fragile, new blood vessels have started to bleed. However, even if bleeding has started, scatter laser treatment may still be possible, depending on the amount of bleeding. If the bleeding is severe, patients may need a surgical procedure called a vitrectomy. During a vitrectomy, blood is removed from the center of the eye. The compositions and methods of the present invention may be effective in treating diabetic retinopathy.

Retinal ischemia occurs when there is a lack of oxygen to the cells of the retina and results in damage or death the retinal cells and consequent loss of vision. Causes include various retinal vascular disorders such as retinal venous occlusion. Hypertension is a risk factor for retinal ischemia. The compositions and methods of the present invention may be effective in treating retinal ischemia.

Retinopathy of Prematurity (ROP), previously known as retrolental fibroplasia, is a disease of the eye that affects premature babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP. The compositions and methods of the present invention may be effective in treating ROP.

Retinal transplantation. The compositions and methods of the invention may be useful in preventing rejection of transplanted retinal tissue. U.S. Provisional Application Nos. 60/880,745 and 60/902,440 (incorporated herein by reference) describe in detail the immunosuppressive properties of the compositions of the invention. Briefly, it has been discovered that extraembryonic cells (EE cells) including but not limited to extraembryonic HLA-G positive cells (EHP cells), TSE cells and AMP cells, and/or cell lysates and/or conditioned media derived therefrom, alone or in combination with each other and/or other suitable active agents, are useful agents capable of treating HVG, GVHD, as well as many other immune and/or inflammatory diseases and disorders. The cells express HLA-G, do not express MHC Class II antigens, are telomerase negative, do not form teratomas, are not immortal, secrete cellular modulatory factors, and are readily available in great numbers.

Obtaining and Culturing of Cells

TSE cells—Various methods for isolating cells from the extraembryonic tissue, which may then be used to produce the TSE cells of the instant invention are described in the art (see, for example, US2003/0235563, US2004/0161419, US2005/0124003, U.S. Provisional Application Nos. 60/666,949, 60/699,257, 60/742,067, 60/813,759, U.S. Application Ser. No. 11/333,849, U.S. Application No. 11/392,892, PCTUS06/011392, US2006/0078993, PCT/US00/40052, U.S. Pat. No. 7,045,148, US2004/0048372, and US2003/0032179).

Identifying TSE cells—Once extraembryonic tissue is isolated, it is necessary to identify which cells in the tissue have the characteristics associated with TSE cells (see definition above). For example, cells are assayed for their ability to secrete VEGF, Angiogenin, PDGF and TGFβ2 and the MMP inhibitors TIMP-1 and/or TIMP-2 into the extracellular space or into surrounding culture media. In some instances, it may be difficult or impossible to detect certain factors using standard assays. This may be because certain factors are secreted by the cells at physiological levels that are below the level of detection by the assay methods. It may also be that the factor(s) is being utilized by the TSE cell and/or by other local cells, thus preventing accumulation at detectable levels using standard assays. It is also possible that the temporal manner in which the factors are secreted may not coincide with the timing of sampling.

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and optionally e) further proliferation of the cells, optionally using additional additives and/or growth factors (i.e. recombinant human EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human fluid or derivative or preparation thereof, such as human serum or amniotic fluid, which contains human protein. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human albumin at a concentration of at least 0.5% and up to 10%. In particular embodiments, the human albumin concentration is from about 0.5 to about 2%. The human albumin may come from a liquid or a dried (powder) form and includes, but is not limited to, recombinant human albumin, PLASBUMIN® normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium , STEMLINE® T-cell expansion medium or STEMLINE® II hematopoietic stem cell expansion medium, OPTI-PRO™ serum-free culture medium, or DMEM culture medium, with human albumin (PLASBUMIN® normal human serum albumin) added up to concentrations of 10%.

The invention further contemplates the use of any of the above basal media wherein animal-derived proteins are replaced with recombinant human proteins and animal-derived serum, such as BSA, is replaced with human serum albumin. In preferred embodiments, the media is serum-free in addition to being animal-free.

Optionally, other factors are used. In one embodiment, epidermal growth factor (EGF) at a concentration of between 0-1 μg/mL is used. In a preferred embodiment, the EGF concentration is around 10-20 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation. All supplements are clinical grade.

Generation of Conditioned Medium

TSE cell conditioned medium—is obtained as described below for ACCS, except that TSE cells are used.

Generation of ACCS—The AMP cells of the invention can be used to generate ACCS. In one embodiment, the AMP cells are isolated as described herein and 10×10⁶ cells are seeded into T75 flasks containing between 5-30 mL culture medium, preferably between 10-25 mL culture medium, and most preferably about 10 mL culture medium. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 3 days post-confluence. In another embodiment the medium is changed and ACCS is collected 4 days post-confluence. In another embodiment the medium is changed and ACCS is collected 5 days post-confluence. In another embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated that ACCS be formulated for sustained-release after collection.

The compositions of the invention can be prepared in a variety of ways depending on the intended use of the compositions. For example, a composition useful in practicing the invention may be a liquid comprising an agent of the invention, i.e. TSE cells, including AMP cells and/or ACCS or PCS, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Pharmaceutical Compositions—The present invention provides pharmaceutical compositions of TSE cells, including AMP cells and/or ACCS or PCS and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits—The invention also provides for an article of manufacture comprising packaging material and a pharmaceutical composition of the invention contained within the packaging material, wherein the pharmaceutical composition comprises compositions of TSE cells, including AMP cells and/or ACCS, or PCS. The packaging material comprises a label or package insert which indicates that the TSE cells, including AMP cells and/or ACCS, or PCS can be used for treating ophthalmic disorders, for example, corneal disorders/diseases/injuries.

Formulation, Dosage and Administration

Compositions comprising TSE cells, including AMP cells and/or ACCS, or PCS may be administered to a subject to provide various cellular or tissue functions, for example, to treat ophthalmic disorders due to trauma, surgery, genetics, disease, etc. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their use in treating ophthalmic disorders or restoring a therapeutically important metabolic function. The compositions may also be administered to the recipient in one or more physiologically acceptable carriers. Carriers for the cells may include but are not limited to solutions of phosphate buffered saline (PBS) or lactated Ringer's solution containing a mixture of salts in physiologic concentrations.

Pharmaceutical compositions useful in the practice of certain embodiments of the invention (i.e. those utilizing topical administration) include a therapeutically effective amount of an active agent with a pharmaceutically acceptable carrier. Such pharmaceutical compositions may be liquid, gel, ointment, salve, slow release formulations or other formulations suitable for ophthalmic administration. The composition comprises a composition of the invention (i.e. TSE cells, including AMP cells and/or ACCS, or PCS) and, optionally, at least one ophthalmically acceptable excipient, wherein the excipient is able to reduce a rate of removal of the composition from the eye by lacrimation, such that the composition has an effective residence time in the eye of about 2 hours to about 24 hours or longer.

In various embodiments, compositions of the invention can comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

Preferably the liquid composition is aqueous. Alternatively, the composition can take form of an ointment. In a preferred embodiment, the composition is an in situ gellable aqueous composition, more preferably an in situ gellable aqueous solution. Such a composition can comprise a gelling agent in a concentration effective to promote gelling upon contact with the eye or lacrimal fluid in the exterior of the eye. Suitable gelling agents non-restrictively include thermosetting polymers such as tetra-substituted ethylene diamine block copolymers of ethylene oxide and propylene oxide (e.g., poloxamine 1307); polycarbophil; and polysaccharides such as gellan, carrageenan (e.g., kappa-carrageenan and iota-carrageenan), chitosan and alginate gums. The phrase “in situ gellable” includes not only liquids of low viscosity that can form gels upon contact with the eye or with lacrimal fluid in the exterior of the eye, but also more viscous liquids such as semi-fluid and thixotropic gels that exhibit substantially increased viscosity or gel stiffness upon administration to the eye or area surrounding the eye.

Aqueous compositions of the invention have ophthalmically compatible pH and osmolality. Preferably these compositions incorporate means to inhibit microbial growth, for example through preparation and packaging under sterile conditions and/or through inclusion of an antimicrobially effective amount of an ophthalmically acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

The composition can comprise an ophthalmic depot formulation comprising an active agent for subconjunctival administration. The ophthalmic depot formulation comprises a composition of the invention (i.e. TSE cells, including AMP cells and/or ACCS, or PCS). The microparticles comprising the compositions can be embedded in a biocompatible pharmaceutically acceptable polymer or a lipid encapsulating agent. The depot formulations may be adapted to release all of substantially all the active material over an extended period of time. The polymer or lipid matrix, if present, may be adapted to degrade sufficiently to be transported from the site of administration after release of all or substantially all the active agent. The depot formulation can be liquid formulation, comprising a pharmaceutical acceptable polymer and a dissolved or dispersed active agent. Upon injection, the polymer forms a depot at the injections site, e.g. by gelifying or precipitating.

The composition can comprise a solid article that can be inserted in a suitable location in the eye, such as between the eye and eyelid or in the conjunctival sac, where the article releases the active agent. Release from such an article is preferably to the cornea, either via lacrimal fluid that bathes the surface of the cornea, or directly to the cornea itself, with which the solid article is generally in intimate contact. Solid articles suitable for implantation in the eye in such fashion generally comprise polymers and can be bioerodible or non-bioerodible. Bioerodible polymers that can be used in preparation of ocular implants carrying a composition in accordance with the present invention include without restriction aliphatic polyesters such as polymers and copolymers of poly(glycolide), poly(lactide), poly(.epsilon.-caprolactone), poly(hydroxybutyrate) and poly(hydroxyvalerate), polyamino acids, polyorthoesters, polyanhydrides, aliphatic polycarbonates and polyether lactose. Illustrative of suitable non-bioerodible polymers are silicone elastomers.

One of skill in the art may readily determine the appropriate concentration, or dose, of the TSE cells, including AMP cells and/or ACCS, or PCS, for a particular purpose. The skilled artisan will recognize that a preferred dose is one which produces a therapeutic effect, such as corneal wound healing, in a patient in need thereof. Of course, proper doses of the TSE cells, including AMP cells and/or ACCS, or PCS, will require empirical determination at time of use based on several variables including but not limited to the severity and type of disease, injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of disease, injury, disorder or condition being treated. In a preferred embodiment, one dose is sufficient. Other preferred embodiments contemplate, 2, 3, 4, or more doses. In other embodiments, the ACCS and/or AMP cells, or PCS may be coated onto the inner surface of a contact lens which is then placed on the eye, thus allowing delivery of ACCS and/or AMP cells, or PCS directly to the ocular surface. The ACCS and/or AMP cells or PCS used for coating the contact lens may be formulated in a liquid, gel or other suitable ophthalmically compatible vehicle.

The present invention provides a method of treating ophthalmic disorders by administering to a subject TSE cells, including AMP cells and/or ACCS, or PCS, in a therapeutically effective amount. By “therapeutically effective amount” is meant the dose of TSE cells, including AMP cells and/or ACCS, or PCS, which is sufficient to elicit a therapeutic effect. Thus, the concentration of TSE cells, including AMP cells and/or ACCS, or PCS, in an administered dose unit in accordance with the present invention is effective in, for example, the treatment of corneal disease/disorder/injury. For treatment of retinal diseases/disorders, doses for injection into the human eye are typically about 50-100 μL/intravitreous injection.

In further embodiments of the present invention, at least one additional neuroprotective agent may be combined with the TSE cells, including AMP cells and/or ACCS, or PCS, to enhance neuroprotection of retinal cells. Such agents include, for example, antioxidants, such as, ascorbate, dimethylthiourea, a-tocopherol and 13-carotene; calcium antagonists, such as, flunarizine; growth factors, such as, basic-FGF, BDNF, CNTF, and IL-1-β; glucocorticoids such as methylprednisolone, dexamethasone; and iron chelators such as desferrioxamine. In addition, it may be desirable to co-administer other agents, including active agents and/or inactive agents, with the TSE cells, including AMP cells and/or ACCS, or PCS, either for treating retinal diseases/disorders or to treat corneal diseases/disorders/injuries. Active agents include but are not limited to cytokines, chemokines, antibodies, inhibitors, antibiotics, anti-fungals, anti-virals, immunosuppressive agents, other cell types, and the like. Inactive agents include carriers, diluents, stabilizers, gelling agents, delivery vehicles, ECMs (natural and synthetic), scaffolds, and the like. When the TSE cells, including AMP cells and/or ACCS, or PCS, are administered conjointly with other pharmaceutically active agents, (i.e., other neuroprotective agents) even less of the TSE cells, including AMP cells and/or ACCS, or PCS, may be needed to be therapeutically effective.

TSE cells, including AMP cells and/or ACCS, or PCS, can be administered by injection into a target site of a subject, preferably via a delivery device, such as a tube, e.g., catheter. In a preferred embodiment, the tube additionally contains a needle, e.g., a syringe, through which the cells and/or ACCS can be introduced into the subject at a desired location. Specific, non-limiting examples of administering cells to subjects may also include administration by intraocular injection, intralesional injection, subconjunctival injection, intravitreal injection, sublimbal injection, retrobulbar injections, intravenous injection, intraarterial injection, intramuscular injection, intrathecal injection, epidural injection, or infusion.

The timing of administration of TSE cells, including AMP cells and/or ACCS, or PCS, will depend upon the type and severity of the ophthalmic disorder being treated. In a preferred embodiment, the TSE cells, including AMP cells and/or ACCS, or PCS, are administered as soon as possible after the ophthalmic disorder is diagnosed. In other preferred embodiments, the TSE cells, including AMP cells and/or ACCS, or PCS, are administered more than one time following diagnosis.

Also contemplated by the methods of the invention are compositions comprising cells that have been partially or fully differentiated from TSE cells, including AMP cells. Such partially or fully differentiated cell compositions are obtained by treating TSE cells, including AMP cells, with appropriate reagents and under appropriate conditions wherein the cells undergo partial or complete differentiation into, for example, retinal cells (i.e. rods cells and/or cones cells), limbal stem cells or corneal epithelial cells. Skilled artisans are familiar with conditions capable of effecting such partial or complete differentiation. The cells may be treated under differentiating conditions prior to use (i.e. prior to transplantation, administration, etc.), simultaneously with use or post-use. In certain embodiments, the cells are treated under differentiation conditions before and during use, during and after use, before and after use, or before, during and after use.

Skilled artisans will recognize that any and all of the standard methods and modalities for treating ophthalmic disorders currently in clinical practice and clinical development are suitable for practicing the methods of the invention. Routes of administration, formulation, co-administration with other agents (if appropriate) and the like are discussed in detail elsewhere herein.

The treatment of ophthalmic disorders can be monitored by employing a variety of tests and measurements including but not limited to standard visual acuity tests, the Amsler Grid Test, fluorescein angiography, optical coherence tomography, and ERG.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Preparation of AMP Cell Compositions.

Amnion epithelial cells were dissociated from starting amniotic membrane using the dissociation agents PXXIII. The average weight range of an amnion was 18-27 g. The number of cells recovered per g of amnion was about 10-15×10⁶ for dissociation with PXXIII.

Method of obtaining selected AMP cells—Amnion epithelial cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in basal medium supplemented with human serum or human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

Generation of ACCS

The AMP cells of the invention can be used to generate ACCS, including pooled ACCS. The AMP cells were isolated as described above and ˜1×10⁶ cells/mL were seeded into T75 flasks containing ˜10 mL culture medium as described above. The cells were cultured until confluent, the medium was changed and ACCS was collected 3 days post-confluence. Optionally, the ACCS is collected again after 3 days, and optionally again after 3 days. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized, irradiated or formulated for sustained-release following collection. It is also contemplated that ACCS be collected at different time points (see Detailed Description for details).

### Example 3

Corneal Epithelial Wound Closure Model.

Rabbits are divided into 3 randomization groups. Animals in each randomization group are pre-dosed 4 times a day with TSE cells, AMP cells, ACCS or unconditioned media in each eye for 1 day before surgery. Group 1 animals received TSE cells in 1 eye and AMP cells in the other eye; group 2 animals received ACCS in 1 eye and unconditioned media in the other eye; and group 3 animals received AMP cells+ACCS in 1 eye and PBS in the other eye. Rabbits undergo a bilateral procedure to remove the full thickness of the central corneal epithelium within a 5-mm trephine mark. After wounding, the eyes are dosed 4 times a day with the same respective predose test articles, and epithelial wound closure is recorded using slit-lamp photography. The data are analyzed to determine the rate of wound closure.

### Example 4

Corneal Trauma Model

TSE cells, AMP cells, and/or ACCS are tested in an animal model of corneal trauma as described in Kim T I, Chung J L, Hong J P, Min K, Seo K Y, Kim E K. Invest Ophthalmol Vis Sci. 2009 October;50(10):4653-9. Because VEGF is secreted by TSE cells, including AMP cells, and is therefore present in ACCS, it is expected that these compositions are useful in corneal healing following trauma.

### Example 5

Bacterial Keratitis Models

TSE cells, AMP cells, and/or ACCS are tested in an animal model of Bacterial keratitis (Pseudomonas) as described in Kwong M S, Evans D J, Ni M, Cowell B A, Fleiszig S M. Infect Immun. 2007 May;75(5):2325-32. TSE cells, AMP cells, and/or ACCS are tested in an animal model of Bacterial keratitis (Staph. aureus) as described in Green S N, Sanders M, Moore Q C 3rd, Norcross E W, et al. Invest Ophthalmol Vis Sci. 2008 January;49(1):290-4.

### Example 6

Herpetic Keratitis Model

TSE cells, AMP cells, and/or ACCS are tested in an animal model of Herpetic keratitis as described in Lambiase A, Coassin M, Costa N, et al. Graefes Arch Clin Exp Ophthalmol. 2008 January;246(1):121-7.

### Example 7

Wound Healing After PRK Model

TSE cells, AMP cells, and/or ACCS are tested in an animal model of wound healing in rabbits after PRK as described in Kaur H, Chaurasia S S, Agrawal V, Wilson S E. Exp Eye Res. 2009 September; 89(3):432-4. In addition, TSE cells, AMP cells, and/or ACCS are tested in an animal model of Wound healing in mice after PRK as described in Mohan R R, Stapleton W M, Sinha S, Netto M V, Wilson S E. Exp Eye Res. 2008 February;86(2):235-40.

### Example 8

Dry Eye Model

TSE cells, AMP cells, and/or ACCS are tested in an animal model of dry eye in rabbits as described in Nagelhout T J, Gamache D A, Roberts L, Brady M T, Yanni J M. J Ocul Pharmacol Ther. 2005 April; 21(2):139-48. In addition, TSE cells, AMP cells, and/or ACCS are tested in an animal model of dry eye in mice as described in Barabino S, Shen L, Chen L, Rashid S, Rolando M, Dana M R. Invest Ophthalmol Vis Sci. 2005 August; 46(8):2766-71 or De Paiva, C. S., et al, Invest Ophthalmol & Vis Sci 2006 47(7):2847-2856).

### Example 9

Therapeutic Potential of TSE Cells, Including AMP Cells, TSE Cell Conditioned Media, ACCS, PCS, and Combinations thereof in Animal Models of Retinal Degeneration.

Numerous review articles are available which provide detailed information on animal models useful for studying retinal degeneration (see, for example, Aguirre, G. and Acland G., (The Dog and Its Genome, © 2006 Cold Spring Harbor Laboratory Press 0-87969-742-3, pp.291-325) review several dog models for studying retinal diseases; The Foundation Fighting Blindness (Animal Models for Studying Inherited Degenerative Retinal Diseases, February 2000) reviews several animal models for studying inherited degenerative retinal diseases; Chader, G. J., (Vision Research 42 (2002) 393-399, Animal models in research on retinal degenerations: past progress and future hope) reviews results obtained with several animal models of retinal degeneration; Hafezi, F., et al., (Br J Ophthalmol 2000; 84:922-927, Molecular Ophthalmology: and update on animal models for retinal degenerations and dystrophies) provide an update on animal models of retinal degeneration; Madan, A and Penn, J. S., (Frontiers in Bioscience 8, d1030-1043, May 1, 2003, Animal Models of Oxygen-Induced Retinopathy) which reviews animal models useful for studying ROP (retinopathy of prematurity); and Paskowitz, D. M., et al., (Br J Ophthalmol 2006;90:1060-1066, Light and Inherited Retinal Disease) which reviews animal models useful for studying light and inherited retinal diseases.

Any or all of the above-referenced animal models which are familiar to skilled artisans are useful for evaluating the therapeutic potential of TSE cells, including AMP cells, TSE cell conditioned media, ACCS, PCS, and combinations thereof in treating retinal diseases and disorders.

Evaluation is performed using, for example, fluorescent angiography, histology and ERG.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is,:

1. A method for treating an ophthalmic disorder, disease or injury in a patient in need thereof comprising administering to the patient a therapeutically effective amount of one or more compositions selected from the group consisting of trophic factor secreting extraembryonic (TSE) cells, conditioned media derived therefrom, cell lysate derived therefrom, cell products derived therefrom, and physiologic cytokine solution (PCS).

2. The method of claim 1 wherein the treatment stimulates proliferation and/or regeneration of corneal epithelial cells.

3. The method of claim 1 wherein the treatment stimulates proliferation and/or activation of corneal stromal fibroblasts.

4. The method of claim 1 wherein the ophthalmic disorder is selected from the group consisting of an ocular surface disorder, a corneal disorder or a lens disorder.

5. The method of claim 4 wherein the ocular surface disorder is selected from the group consisting of keratitis, corneal ulcers and corneal wounds.

6. The method of claim 5 wherein the corneal wounds are selected from the group consisting of chemical wounds, thermal wounds, surgical wounds and mechanical wounds.

7. The method of claim 5 wherein the keratitis is caused by amoebic, bacterial, fungal or viral infection; photokeratitis; exposure (eyelid dysfunction); chemical injury; trauma; surgery;
keratoconus; Fuchs' dystrophy; or keratoconjunctivitis sicca.

8. The method of claim 7 wherein the surgery is selected from the group consisting of laser-assisted in situ keratomileusis (LASIK), photorefractive keratectomy (PRK), cataract, corneal transplant and pterygium surgery.

9. A method for reducing the risk for corneal transplant rejection in a patient in need thereof comprising administering to the patient a therapeutically effective amount of one or more compositions selected from the group consisting of TSE cells, conditioned media derived therefrom, cell lysate derived therefrom and cell products derived therefrom.

10. The method of claim 1 or 9 wherein the TSE cells are Amnion-derived Multipotent Progenitor (AMP) cells.

11. The method of claim 1 or 9 wherein the conditioned medium is Amnion-derived Cellular Cytokine Solution (ACCS).

12. The method of claim 11 wherein the ACCS is formulated for sustained-release.

13. The method of claim 1 or 9 wherein the TSE cells, conditioned media derived therefrom, cell lysate derived therefrom or cell products derived therefrom are administered in combination with other agents or treatment modalities.

14. The method of claim 13 wherein the other agents are active agents.

15. The method of claim 14 wherein the active agents are selected from the group consisting of growth factors, cytokines, inhibitors, immunosuppressive agents, steroids, chemokines, antibodies, antibiotics, antifungals, antivirals, mitomycin C, and other cell types.

16. The method of claim 15 wherein the other treatment modalities are selected from the group consisting of bandage contact lens, fibrin glue, tarsorraphy (partial suturing of the eyelids), autologous serum, Gunderson flap and corneal transplant.
